(19) United States
(12) Patent Application Publication
(10) Pub. No.: US 2007/0156453 A1
(43) Pub. Date: Jul. 5, 2007

(54) INTEGRATED TREATMENT PLANNING SYSTEM

(75) Inventors: Nils Frielinghaus, Heimstetten (DE); Robert Schmidt, Munich (DE); Christoph Pedain, Munich (DE)

Correspondence Address: DON W. BULSON (BrainLAB), RENNER, OTTO, BOISSELLE & SKLAR, LLP, 1621 EUCLID AVENUE - 19TH FLOOR, CLEVELAND, OH, 44115, US

(73) Assignee: BRAINLAB AG, Feldkirchen (DE)

(21) Appl. No.: 11/539,683

(22) Filed: Oct. 9, 2006

(30) Foreign Application Priority Data
Oct. 7, 2005 (EP) 05021932.8

(51) Int. Cl. A61N 5/10; G06Q 10/00; G06Q 50/00

(52) U.S. Cl. 705/2

(57) ABSTRACT

A medical treatment planning system includes a computer including a processor and memory, data stored in the memory, said data including a priori of information relating to medical conditions, medical treatments and treatment results, and treatment planning logic stored in the memory and executable by the processor. The treatment planning logic includes logic that obtains pre-treatment patient data describing the patient's medical condition, logic that analyzes the pre-treatment patient data relative to the a priori of information and, based on the analysis, formulates a first treatment plan for treating the patient, and logic that outputs the first treatment plan for evaluation by medical personnel.

### FIELD OF THE INVENTION

The present invention relates to planning systems and, more particularly, to an integrated treatment planning system and method.

### BACKGROUND OF THE INVENTION

Single solutions for tumor treatment planning systems exist. For example, three main therapies are used to treat brain tumors: resection, energy (e.g., radiation, heat, or light), and drug delivery. There are cases, however, wherein single solutions provide success rates that are less than optimal (e.g., after treatment, the tumor may persist).

Most treatment methods can be separated into two steps: pre-treatment planning and the actual treatment. During pre-treatment planning, patient data may be obtained and/or analyzed to formulate the treatment plan, while during actual treatment, the formulated plan is executed. Due to the treatment, the patient's anatomy and/or physiology may be altered, e.g., by resecting a tumor mass. Thus, in order to take into account these changes in the patient, new patient data may be obtained in real time (e.g., a new CT scan, MR scan, etc.) prior to a further treatment.

Coordinating two or more cancer treatment plans, for example, to achieve a best possible success rate can be challenging. This is due in part to the fact that treatment success rates are influenced by multiple factors that can contradict each other. For example, cancer treatment often involves radical treatment wherein tumor cells are destroyed wherever they occur. This destruction, however, can affect the quality of life during and after the treatment procedures.

In addition, treatment planning is becoming more and more complicated as additional treatments for partial aspects of a disease become available (e.g., pain, tissue swelling, etc.) and as additional treatments are utilized that have only a limited impact on the disease but still cause side effects (e.g., treatments to which only certain cell types of a tumor respond).

### SUMMARY OF THE INVENTION

The invention provides a system and method that enables automatic generation of a medical treatment plan, wherein the plan is based on patient information as well as a priori information relating to the disease, treatments, and treatment results.

According to one aspect of the invention, there is provided a medical treatment planning system that provides a recommended course of action for treating a patient. The planning system includes a computer having a processor and memory, and data stored in the memory. The data includes a priori information relating to medical conditions, medical treatments and treatment results. The system also includes treatment planning logic stored in the memory and executable by the processor. The treatment planning logic includes logic that accepts pre-treatment patient data describing the patient's medical condition, and logic that analyzes the pre-treatment patient data relative to the a priori information and, based on the analysis, formulates a first treatment plan for treating the patient. The treatment plan then is output or otherwise provided to medical personnel.

Further, the medical treatment planning system can report certain risk factors based on specific criteria. For example, a patient with a certain disease may be treated a number of different ways. The treatment planning system can analyze the different treatment approaches and provide data with respect to the expected life span, quality of life, side effects, etc. for each treatment approach. The medical personnel and/or patient then may be provided these results, and a decision can be made on how to treat the disease.

To the accomplishment of the foregoing and related ends, the invention, then, comprises the features hereinafter fully described and particularly pointed out in the claims. The following description and the annexed drawings set forth in detail certain illustrative embodiments of the invention. These embodiments are indicative, however, of but a few of the various ways in which the principles of the invention may be employed.

### BRIEF DESCRIPTION OF THE DRAWINGS

The forgoing and other embodiments of the invention are hereinafter discussed with reference to the drawings.

FIG. 1 is a block diagram illustrating an exemplary system for treatment planning in accordance with the invention.

FIG. 2 is a block diagram of an exemplary database in accordance with the invention.

FIGS. 3A-3C illustrate exemplary tables within the database of FIG. 2 in accordance with the invention.

FIG. 4 is a flow chart illustrating exemplary steps for integrating treatment planning in accordance with the invention.

### DETAILED DESCRIPTION

While the invention is primarily described with respect to treating cancers, it will be appreciated that the invention may be applied to other diseases, and reference to treating cancers is not intended to be limiting in any way.

Many paths can be chosen to structure the variety of treatments and/or to classify certain methods of therapy or diagnosis. One particularly useful way of classifying today's tumor therapies, for example, is presented below, and most tumor treatments applied to patients today contain elements of the following four classes:
1. Surgical resection;
2. Therapeutic application of energy (e.g., “radiosurgery”, “radiotherapy”, “thermotherapy”, “light therapy”);
3. Application of therapeutic drugs (e.g., “chemotherapy”, “immunotherapy”, “targeted chemotherapy” or even “personalized chemotherapy”)—whereby the application of drugs can either be systemic or local; and
4. Diagnostic procedures (e.g., with medical images, imaging tracers, or targeted diagnostics) and supportive care (e.g., reduction of pain or tissue swelling).
   To phrase the matter differently, there are many ways to treat a particular disease, such as a tumor, and every particular tumor generally needs (a combination of) many treatments.

Provided herein is a system and method that utilizes a priori information in conjunction with patient parameters to provide one or more treatment plans. The a priori information can include data regarding different medical conditions or diseases, including data regarding previous subjects having similar symptoms, recommended treatments of such diseases, and actual and/or expected results of such treatments.

Further, the treatment planning system may provide or report certain factors of interest to the medical personnel and/or patient. These factors can be based on the patient parameters in conjunction with the a priori information and can include, for example, risk factors associated with the disease and/or treatment procedure, likelihood that the disease will be cured, likely side effects, long and short term effects, quality of life, etc. These factors, which, for example, may be reported in the form of probabilities, can be used by the medical personnel and/or patient to determine how to proceed in treating the disease.

For example, a patient with a particular disease may be given two or more different options for treating the disease. The first option may have a ninety percent likelihood of extending the patient's life 12-15 months, while the second option may have a seventy-five percent likelihood of extending the patient's life 12-15 months. From this stand point alone, it would appear that option 1 is better than option 2. However, this may not be the case when other factors are considered. In particular, option 1 may be more likely than option 2 to require significant bed rest after the procedure is performed. Further, even though option 1 is more likely to provide a 12-15 month life span, it also may be more likely than option 2 to provide discomfort during the last several months of that life span. When presented with these probabilities, the patient may elect option 2 over option 1, even though he/she is less likely to reach the 12-15 month life expectancy with option 2.

Further, after the first treatment is performed, data can be collected for or at a predetermined time after the first procedure. This data then can be used to plan a second treatment, wherein the combination of the first and second treatment can provide improved exploitation of patient data. This data can be measured directly or derived from other data, such as the measured data, for example. The measurements can include, for example, data obtained from medical imaging (such as from Magnetic Resonance Imaging, Computed Tomography imaging, PET, SPECT, x-ray, and/or ultrasound). In addition to imaging data, measurements can include any other physiological property such as blood values or Karnofski performance status of a patient.

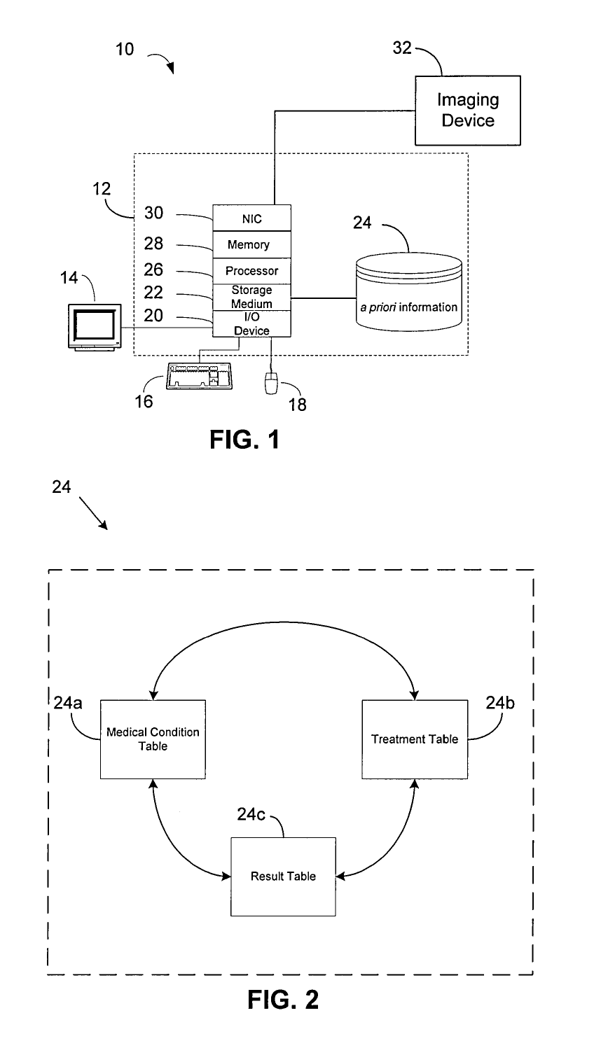

To this end, there is provided a method and a device for loading, displaying, and analyzing patient data. FIG. 1 is a block diagram of an exemplary system 10 for implementing treatment planning in accordance with the invention. The system 10 includes a computer 12 for processing data, and a display 14 for viewing system information. A keyboard 16 and pointing device 18 may be used for data entry, data display, screen navigation, etc. The keyboard 16 and pointing device 18 may be separate from the computer 12 or they may be integral to it. A computer mouse or other device that points to or otherwise identifies a location, action, etc., e.g., by a point and click method or some other method, are examples of a pointing device. Alternatively, a touch screen (not shown) may be used in place of the keyboard 16 and pointing device 18. The display 14, keyboard 16 and mouse 18 communicate with the computer 12 via an input/output device 20, such as a video card and/or serial port (e.g., a USB port or the like).

Included in the computer 12 is a storage medium 22 for storing information, such as application data, screen information, programs, etc., part of which may be in the form of a database 24. The storage medium 22 may be a hard drive, for example, or any other storage means that can retain data, including magnetic, optical, etc. A processor 26, such as an AMD Athlon 64® processor or an Intel Pentium IV@ processor, combined with a memory 28 and the storage medium 22 execute programs to perform various functions, such as data entry, numerical calculations, screen display, system setup, etc. The memory 28 may comprise several devices, including volatile and non-volatile memory components. Accordingly, the memory 28 may include, for example, random access memory (RAM), read-only memory (ROM), hard disks, floppy disks, optical disks (e.g., CDs and DVDs), tapes, flash devices and/or other memory components, plus associated drives, players and/or readers for the memory devices. The processor 26 and the memory 28 are coupled using a local interface (not shown). The local interface may be, for example, a data bus with accompanying control bus, a network, or other subsystem.

A network interface card (NIC) 30 allows the computer 12 to communicate with devices external to the system 10. A medical imaging device 32, such as, for example, a Magnetic Resonance Imaging device, Computed Tomography imaging device, PET, SPECT, x-ray, ultrasound, or the like, may be communicatively coupled to the system 10 via the NIC 30. The medical imaging device 32 can provide patient specific data that can be used by the computer 12 in making treatment recommendations, for example.

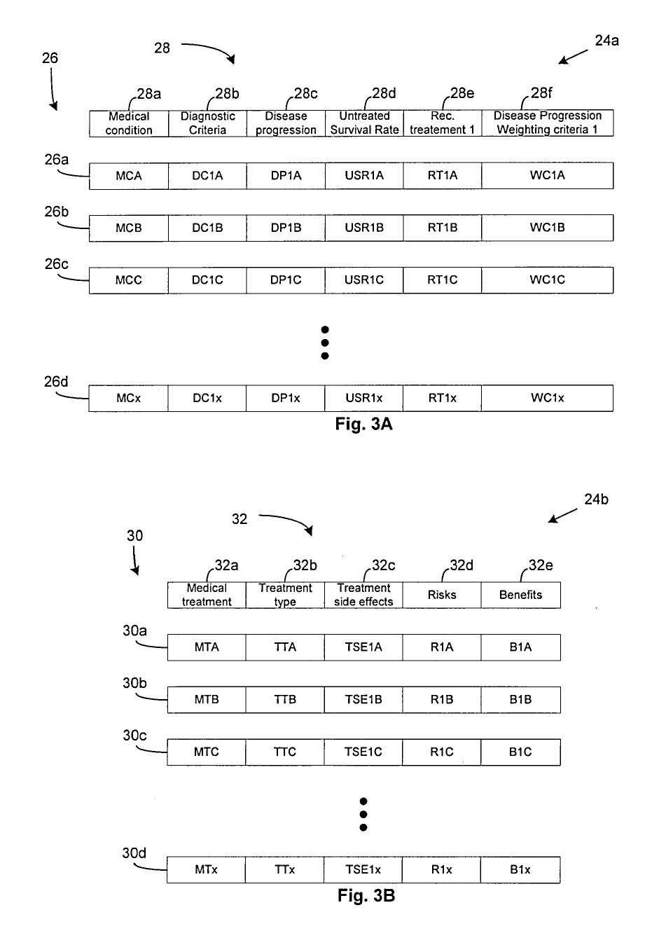

Moving now to FIG. 2, the database 24 will be described in more detail. It should be appreciated that the following description of the database 24 is merely exemplary, and it may take on other forms without departing from the scope of the invention. Further, the data stored in the database may be numerical, textual, graphical, etc.

The database 24, which may be a relational database, for example, can include multiple tables, such as a medical condition table 24a, a treatment table 24b, and a result table 24c. More or fewer tables may be implemented without departing from the scope of the invention.

The medical condition table 24a, for example, includes data pertaining to different medical conditions, such as different types of cancer (e.g., brain cancer, stomach cancer, lung cancer, etc.), different types of heart problems (e.g., heart disease), etc. While each of these different ailments are described and shown in a single table, it will be appreciated that each disease may be broken out into different sub tables within the main medical condition table 24a. For example, a first sub-table may be dedicated to brain tumors, wherein different medical conditions within the table pertain to a location and/or size of the tumor in the brain. A second sub-table may be dedicated to lung cancer, wherein different medical conditions within the second sub-table pertain to a size and/or location of cancerous tissue in the lungs. The medical condition table 24a may include additional information, such as life expectancy if the disease is untreated, expected progression of the disease, expected symptoms during disease progression, as well as weighting factors based on various considerations, such as the age of the patient, etc.

The treatment table 24b can include various treatments for a particular disease. For example, a brain tumor located at a particular region in the brain, based on previous experience, may have a first recommended or preferred treatment (e.g., surgery), while a brain tumor in another region of the brain may have a second preferred or recommended treatment (e.g., surgery followed by radiation therapy). Further, the treatment table 24b may include data pertaining to side effects of the each treatment, the risks associated with a particular treatment, the benefits associated with a particular treatment, etc. The treatment table 24b also may include weighting factors as noted above for the medical condition table 24a.

The results table 24c can include the expected results for a particular medical condition treated with a particular treatment. For example, previous experience may show that a particular disease, such as a brain tumor, for example, when treated with surgery has an identifiable likelihood of success (e.g., described as a probability of success). When that same disease is treated with radiation therapy, there also is an identifiable likelihood of success, which may be different from that obtained via surgery (e.g., a different probability). Further, when the two methods are combined, they may provide another result that is the same, more likely, or less likely to produce beneficial results than the two methods standing alone. The results table 24c also may include statistics relating to the quality of life the individual can expect to achieve, wherein the quality of life is based on the medical condition, the treatment plan, and the patient parameters (e.g., progression of disease at time of treatment, etc.).

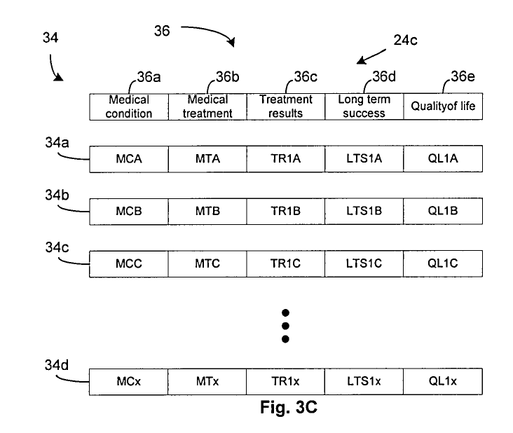

With further reference to FIGS. 3A-3C, exemplary structures of the respective tables 24a, 24b and 24c are shown. It is noted that for sake of clarity, the tables only show a single column for a particular category or entry (e.g., a single symptom column, a single disease progression column, etc.). However, in practice there may be multiple columns describing various criteria for a particular medical condition. For example, there may be multiple symptoms or “diagnostic criteria” associated with a particular medical condition. To accommodate for such possibilities, there may be multiple columns to identify different criteria (e.g., first, second and third diagnostic criteria columns).

FIG. 3A illustrates an exemplary medical condition table 24a including a plurality of rows 26a-26d (referred to generally as rows 26) and columns 28a-28f (referred to generally as columns 28), wherein an intersection of a row and column defines a data storage cell. A medical condition column 28a defines a particular medical condition, e.g., a particular disease, such as a brain cancer, lung cancer, etc. The particular disease can be entered in the cell corresponding to the intersection of the medical condition column 28a and any row 26. For example, the intersection of the first row 26a and the medical condition column 28a defines a first cell that may store information relating to a first medical condition (e.g., a particular type of brain cancer). The intersection of the second row 26b and the first medical condition column 28a defines a second cell that may store information relating to a second medical condition (e.g., lung cancer). Then, each entry within the respective rows 26a and 26b pertain to the medical condition defined in the first cell of that row (e.g., all data in the first row 26a pertains to a patient with brain cancer). This convention is followed through the exemplary tables provided herein.

A diagnostic criteria column 28b can describe one or more diagnostic criteria typical of a disease (e.g., headaches, seizures, imaging data, etc.), wherein a diagnostic criterion is entered in a cell corresponding to the particular medical condition, e.g., diagnostic criterion 1B (DC1B) is entered in the cell defined by the row corresponding to medical condition B (MCB).

A disease progression column 28c identifies data relating to expected progression of the disease (if left untreated). Additional disease progression columns (not shown) may describe expected disease progression based on different treatments of the disease, age factors, etc. An untreated survival rate column 28d describes data corresponding to the expected survival rate of the patient with the particular medical condition (if untreated). This entry my be based on various patient information, including age, sex, etc.

A recommended treatment column 28e provides a recommended treatment plan for the corresponding medical condition. For a brain tumor, for example, the recommended treatment may be surgery followed by radiation therapy. The recommended treatment plan can be based on experience and/or actual results obtained with similar patients experiencing similar medical conditions.

In addition to the above, weighting criteria may be added to the specific table entries. There may be a particular level of certainty with respect to each entry, which may be entered into the table 24a. For example, there may be a 70% probability that the disease described in the medical condition column 28a will progress as described in the disease progression column 28c. This probability can be entered into the table in weighing criteria column 28f. Although not shown, there may be weighting criteria for other entries in the table 24a.

Moving now to FIG. 3B, there is shown an exemplary treatment table 24b that includes rows 30a-30d (designated generally as rows 30) and columns 32a-32e (designated generally as columns 32). As described above, the intersection of a row and column defines a data storage cell.

The treatment table 24b includes a medical treatment entry 32a, which describes a particular medical treatment or procedure. For example, the medical treatment may be a particular type of surgery, radiation therapy, drug therapy, etc. A treatment type column 32b can describe aspects of the medical treatment corresponding to the entered medical treatment. For example, if the medical treatment entered in the cell defined by the intersection of the first row 30a and the medical treatment column 32a is radiotherapy, radiosurgery, thermotherapy, or light therapy, then the treatment type entered in the cell defined by the intersection of the first row 30a and the treatment type column 32b may be therapeutic application of energy. Other treatment types may be surgical, application of therapeutic drugs (e.g., chemotherapy, immunotherapy, targeted chemotherapy, etc.), and diagnostic procedures and other supportive care (e.g., diagnostic images, reduction of pain and swelling, etc.).

The treatment table 24b also can include a treatment side effects column 32c. Data cells corresponding to the side effects column 32c describe the side effects associated with a particular medical treatment (i.e., the medical treatment described in the corresponding cell of the medical treatment column 32a). The treatment table 24b also may include risk column 32d and benefit column 32e. Data cells corresponding to these columns can describe the specific risks and benefits associated with the particular medical procedure. Further, and although not shown, weighting criteria as described above in the medical condition table 24a also may be implemented in the medical treatment table 24b (e.g., likelihood a particular risk or benefit will be realized).

FIG. 3C illustrates an exemplary results table 24c that includes a plurality of rows 34a-34d (referred to generally as rows 34) and a plurality of columns 36a-36e (referred to generally as columns 36). As above, the intersection of a row and column defines a cell for storing information.

A medical condition column 36a corresponds to the medical condition column 28a of medical condition table 24a, while a medical treatment column 36b corresponds to the medical treatment column 32a of the treatment table 24b. Further, a treatment results column 36c describes expected treatment results when the particular treatment identified in the treatment entry 36b is used to treat the condition described in the medical condition entry 36a. For example, the expected results may be that the patient is completely cured, or the patient will need to have followup treatments, or the treatment results may be dependent on other factors not yet determined (e.g., how much of a tumor was removed). In a similar manner, a long term success column 36d and a quality of life column 36e can provide data that identifies a likelihood that the patient will be disease free after a predefined period of time, and how the patient feels and/or how much care may be required subsequent to treatment.

Like the two tables above, the results table 24c also may include weighting factors indicating probabilities that conditions described in the tables are likely to occur.

Data contained within the database 24, for example, can be collected from previous experience with a plurality of patients, diseases associated with those patients, how the diseases were treated, and the results obtained from the treatment. Techniques for obtaining data that can be used in the database 24 may be found in co-pending application Ser. No. 11/419,535 filed May 22, 2006, the contents of which is incorporated by reference in its entirety.

In practice, patient specific data is entered into the system 10. The patient specific data can include biographic data (e.g., age, height, weight, sex, etc.) and diagnostic data (e.g., data obtained from diagnostic tests, imaging data and/or physical observations). Once the data is entered, the system 10 proceeds to search the database 24 for data that matches or is similar to the patient data. Information that matches or is substantially similar to the key information can be extracted from the database 24 and used to form the basis of a report.

For example, the patient data may comprise the patient's current medical data, including age, height, weight, diagnostic data (e.g., diagnostic test results, imaging data), data derived from such medical data, etc. The data can form the basis for a query into the database 24, and the results of the query can form the basis of the report. The report can include information such as the likelihood of success of the medical treatment, possible complications, expected quality of life, risk factors and their likelihood of occurring, etc. This report then can be presented to medical personnel and/or the patient and, based on the report, a decision can be made on how to treat the disease.

Further, simulations may be run for one or more treatment plans, wherein the simulations can provide data corresponding to how a disease may respond and/or progress after treatment. For example, two or more different treatments may be simulated for a particular patient (using the patient data), and the effects of the respective treatments may be communicated as a visual representation on the display 14, prior to actual implementation of the treatment. Additionally, weighting criteria for data in the database 24 may be altered, and the effects shown on the display.

The database 24 can be continuously updated with new data so as to reflect the latest medical advances. In this sense, the system 10 is adaptive as it can be continuously updated with the latest medical information.

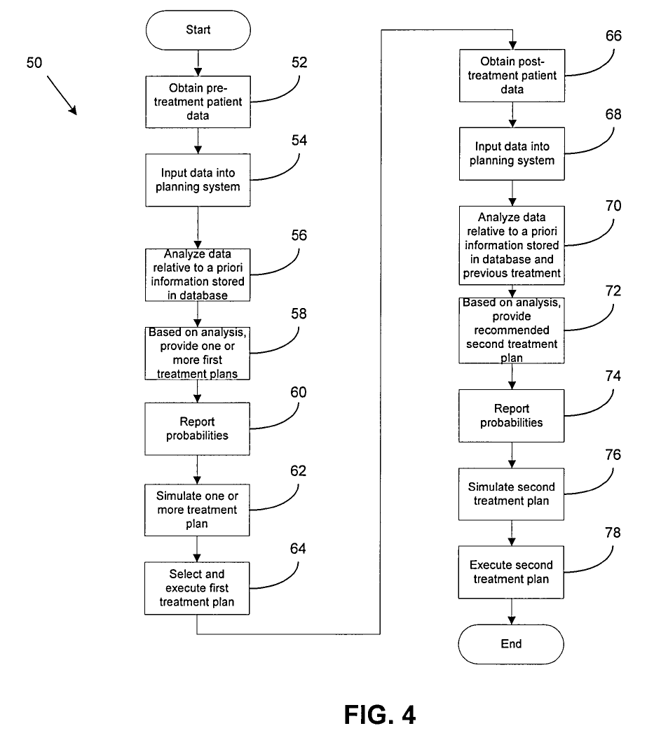

Moving now to FIG. 4, there is provided a flow chart 50 illustrating exemplary steps that may be performed in carrying out treatment planning. The flow chart includes a number of process blocks arranged in a particular order. As should be appreciated, many alternatives and equivalents to the illustrated steps may exist and such alternatives and equivalents are intended to fall with the scope of the claims appended hereto. Alternatives may involve carrying out additional steps or actions not specifically recited and/or shown, carrying out steps or actions in a different order from that recited and/or shown, and/or omitting recited and/or shown steps. Alternatives also include carrying out steps or actions concurrently or with partial concurrence.

Beginning at block 52, pre-treatment patient data is obtained. Pre-treatment patient data can include, for example, data describing the patient's current condition, including diagnostic data (e.g., medical imaging, blood work, etc.) as well as biographical data (e.g., age, weight, height, sex, etc.). The data can be obtained by performing various diagnostic tests and/or scans (e.g., MRI, CT, etc.) on the patient.

At block 54, the patient data is input or otherwise loaded into the computer 12. Data input may be manual (using the keyboard to enter data), semi automatic (e.g., indicating to the computer a location of the data and command the computer to load the data), or automatic (e.g., direct input of the data from an external device via the NIC 30). Data loading may not only entail simple reading of information from a data repository such as a hard drive, or collecting data from a user input mask on a computer screen. Data loading can include pre-processed data in order to create derivatives from that information. These derivatives, for example, can be computed from the combination of various input images. For instance, the position of nerve fibers in the brain can be computed from Magnetic Resonance Imaging, in particular Diffusion Tensor MRI. Other derivatives may be built from a combination of measurements with data that is stored in databases, such anatomical or functional atlases or other generalized information such as tissue elasticities or permeabilities. Alternatively, input information can be computed from accessing time series of information about a particular patient or about generalized disease and/or therapy databases.

At block 56, the patient data is analyzed relative to a priori information stored in the database 24. More specifically, the patient specific data can be used to search the database 24 for patients with similar conditions and characteristics. Data for matching or similar patients then can be extracted to determine how these patients were treated and the results that were obtained. At block 58, the extracted patient data can be used to plan or recommend a treatment for the instant patient. The treatment plan may include one or more of surgical treatment, treatment using the delivery of energy, or treatment using drugs. Further, the treatment plan can combine multiple treatment methods. For example, the application of radiation (energy) can be planned for in conjunction with the application of radiation sensitizers (drugs).

At block 60, data corresponding to results of the treatment plan also is provided to the medical personnel and/or patient. This data, which can be presented in the form of probabilities, can be based on how close the patient data matches the data within the database 24 and/or weighting factors for the specific entries in the database 24. At block 62, the treatment plan or plans may be simulated to provide feedback as to the probable effectiveness of the treatment plan. Based on the recommended treatment plan and/or simulation results, a treatment is selected and executed as indicated at block 64.

At block 66, the progress of the treatment is monitored and data is collected. Monitoring the progress and/or outcome of the first treatment and/or collecting data can include observing the physical, anatomical, functional, or physiological changes caused by the treatment. Again, in some cases monitoring may be as simple as looking at an image from a scanner. In many cases, however, proper assessment of the true outcome of a treatment will depend on the comprehensive analysis of a variety of input information. The analysis often requires massive computation, including the computation on medical images to make them comparable over time. In the case of a tumor treatment, for example, medical images often only show a true distinction between success of a treatment and recurrence of the tumor when they are regarded over time. A good analysis of time series of patient image data includes co-registration of the most relevant images acquired before and after the treatment. Due to the disease progression and/or the treatment effect, the anatomical situation may change during the various measured time points, and elastic registration (“morphing”) of images may be required. These methods then allow for the correlation of the precise shape of anatomical structures, functional areas, physiological information, or disease information. Once collected, then at block 68 the data is input into the system 10 as described above with respect to block 54.

At block 70, the post-treatment patient data is analyzed relative to a priori information stored in the database 24 as described above with respect to block 56. Then, at block 72, the analysis is used to plan a subsequent treatment, wherein the subsequent treatment can include one or more of the previously mentioned treatment methods (surgery, deliver of energy, delivery of drugs). At block 74, data corresponding to likely results is provided (e.g., probabilities as described in block 60), and at block 80, the second treatment plan or plans may be simulated. Based on the recommended treatment plan and/or simulation results, a second treatment can be selected and executed as indicated at block 82.

The actual code for performing the functions described herein can be readily programmed by a person having ordinary skill in the art of computer programming in any of a number of conventional programming languages based on the disclosure herein. Consequently, further detail as to the particular code itself has been omitted for sake of brevity. The computer code and/or databases may be embodied on a machine (e.g., computer) readable medium, such as a magnetic, optical or electronic storage device (e.g., hard disk, optical disk, flash memory, etc.).

Accordingly, medical conditions can be treated using a priori information relating to past medical experience. Further, using information collected prior to, during and after the treatment process, the overall plan becomes much more comprehensive and “smarter”, since at the time a subsequent treatment is planned, information about the effectiveness of the previous treatment is known. It is noted that the analysis of image information reveals not only an overall effect or effectiveness of the treatment (or in terms of the disease, not only an overall information about stability, progression or regression), but also locally varying information. This locally varying information can now be correlated with the knowledge about the locally varying application of treatments (e.g., the precise treatment volumes for radiation therapy or radiation surgery). The combined information provides a much better basis for planning a subsequent treatment than a new “snapshot” at the time of re-treatment that presently is used.

Thus, planning of various treatments can be integrated by monitoring the execution of one or a multitude of treatments in light of not only the patient data collected at the time of treatment, but by comprehensive analysis of a wealth of information about the particular patient and the particular disease, generalized information, and also data collected in preceding treatment cycles.

Treatments were previously performed in a disjunctive manner hence not allowing a systematic and/or automatic correlation of all available patient and treatment data. As disclosed herein, the gap between diagnosis, treatment, repeat diagnoses and repeat treatments is closed, in particular by providing a platform for interdisciplinary cross-hospital data exchange and treatment management.

Although the invention has been shown and described with respect to a certain preferred embodiment or embodiments, it is obvious that equivalent alterations and modifications will occur to others skilled in the art upon the reading and understanding of this specification and the annexed drawings. In particular regard to the various functions performed by the above described elements (components, assemblies, devices, compositions, etc.), the terms (including a reference to a “means”) used to describe such elements are intended to correspond, unless otherwise indicated, to any element which performs the specified function of the described element (i.e., that is functionally equivalent), even though not structurally equivalent to the disclosed structure which performs the function in the herein illustrated exemplary embodiment or embodiments of the invention. In addition, while a particular feature of the invention may have been described above with respect to only one or more of several illustrated embodiments, such feature may be combined with one or more other features of the other embodiments, as may be desired and advantageous for any given or particular application.

What is claimed is:

1. A medical treatment planning system that provides a recommended course of action for treating a patient, comprising:
a computer including a processor and memory;
data stored in the memory, said data including a priori of information relating to medical conditions, medical treatments and treatment results; and
treatment planning logic stored in the memory and executable by the processor, said treatment planning logic including: logic that obtains pre-treatment patient data describing the patient's medical condition; logic that analyzes the pre-treatment patient data relative to the a priori of information and, based on the analysis, formulates a first treatment plan for treating the patient; and logic that outputs the first treatment plan for evaluation by medical personnel.

2. The treatment planning system of claim 1, wherein the treatment planning logic further comprises:
logic that accepts post first treatment patient data regarding actual results, conditions and/or physical changes of the patient, said post first treatment patient data obtained a predetermined time period after implementation of the first treatment plan;
logic that analyzes the post first treatment patient data relative to the a priori information and, based on the analysis, formulates a second treatment plan for treating the patient; and
logic that outputs the second treatment plan for evaluation by medical personnel.

3. The treatment planning system of claim 1, wherein the first treatment plan includes at least one of surgery, energy delivery, or drug delivery.

4. The treatment planning system according to claim 3, wherein the second treatment plan includes at least one of surgery, energy delivery, or drug delivery.

5. The treatment planning system of claim 1, wherein the treatment logic further comprises logic that simulates results of the first treatment plan prior to implementation of the first treatment plan.

6. The treatment planning system of claim 1, wherein the treatment logic further comprises logic that models a progression of diseased tissue of the patient.

7. The treatment planning system of claim 1, further comprising at least one interface for inputting and outputting data to/from the computer.

8. The treatment planning system of claim 1, wherein the a priori of information relating to medical conditions includes data regarding at least one of disease symptoms, untreated survival rates of the disease, rate of disease progression, disease side effects, or recommended treatments.

9. The treatment planning system of claim 1, wherein the a priori of information relating to medical treatments includes data regarding at least one of treatment type, treatment side effects, treatment risks, or treatment benefits.

10. The treatment planning system of claim 9, wherein treatment type includes at least one of surgery, energy delivery, or drug delivery.

11. The treatment planning system of claim 9, wherein treatment side effects includes at least one of physical and/or emotional pain or suffering, or expected tissue swelling.

12. The treatment planning system of claim 1, wherein the a priori of information relating to treatment results includes data regarding treatment success rate, quality of life.

13. A computer program embodied on a computer readable medium for providing a recommended course of action for treating a patient, comprising:
data including a priori of information relating to medical conditions, medical treatments and treatment results; and
code that obtains pre-treatment patient data describing the patient's medical condition;
code that analyzes the pre-treatment patient data relative to the a priori of information and, based on the analysis, formulates a treatment plan for treating the patient; and
code that outputs the treatment plan for evaluation by medical personnel.
